(19) United States
(12) Patent Application Publication
(10) Pub. No.: US 2025/0152152 A1
(43) Pub. Date: May 15, 2025

(54) INVERTED SURGICAL RETRACTOR DEVICE

(71) Applicant: Harry Newman-Plotnick, Albany, NY (US)

(72) Inventor: Harry Newman-Plotnick, Albany, NY (US)

(21) Appl. No.: 18/947,815

(22) Filed: Nov. 14, 2024

Related U.S. Application Data
(60) Provisional application No. 63/548,459, filed on Nov. 14, 2023.

(51) Int. Cl. A61B 17/02; A61B 17/00

(52) U.S. Cl. CPC A61B 17/0206; A61B 2017/00747

(57) ABSTRACT

A surgical apparatus is disclosed. A surgical retractor comprises a handle with a central axis; a shaft along the central axis with a distal portion; and a head distal to the shaft, the head having at least one hook oriented such that an end of the at least one hook faces a direction opposite that of the handle, wherein the head runs along a parallel axis to the central axis and is offset from the central axis.

### CROSS-REFERENCE TO RELATED APPLICATIONS

This application claims priority from U.S. Provisional Application No. 63/548,459, titled “INVERTED SURGICAL RETRACTOR DEVICE,” filed on Nov. 14, 2023, the entire contents of which are hereby incorporated by reference.

### TECHNICAL FIELD

The disclosure is generally directed to a surgical tool, and in particular, an ergonomically designed hook retractor tool.

### BACKGROUND OF THE DISCLOSURE

Current surgical practices require the use of retractors, wherein a surgical assistant pulls back an opening to expose a surgical site. The pulling action performed by the surgical assistant over the course of the surgery can be exhausting and difficult.

There is a need in the art for an ergonomically designed retractor that minimizes user discomfort without a decrease in performance. Accordingly, the present disclosure describes an inverted retractor designed to push tissue away from the user.

### SUMMARY

According to certain aspects of the present disclosure, devices are disclosed for the ergonomic design of a surgical tool.

In certain embodiments, a surgical retractor comprises a handle with a central axis; a shaft along the central axis with a distal portion; and a head distal to the shaft, the head having at least one hook oriented such that an end of the at least one hook faces a direction opposite that of the handle, wherein the head runs along a parallel axis to the central axis and is offset from the central axis.

In some embodiments, the head comprises two hooks.

In some embodiments, the two hooks are spaced equidistant from the parallel axis.

In some embodiments, the end of the at least one hook is pointed.

In some embodiments, the end of the at least one hook is blunt.

In some embodiments, the head is offset to the left of the shaft.

In some embodiments, the head is offset to the right of the shaft.

In some embodiments, the head is offset anterior to the shaft.

In some embodiments, the distal portion is a square.

In some embodiments, the distal portion is curved.

In some embodiments, the distal portion is a 180° arc.

In some embodiments, each hook of the at least one or more hooks is a 180° arc.

In other embodiments, a method of using a surgical retractor comprises attaching a surgical retractor to a tissue at an incision site, the surgical retractor comprising: a handle with a central axis; a shaft along the central axis with a distal portion; and a head distal to the shaft, the head having at least one hook oriented such that an end of the at least one hook is oriented to face a direction opposite that of the handle, wherein the head runs along a parallel axis to the central axis and is offset from the central axis; pushing the surgical retractor distally, thereby retracting the tissue away from the incision site; and securing the tissue in a retracted position away from the incision site.

In some embodiments, each hook of the at least one hook has a sharpened end, and attaching the surgical retractor comprises piercing the tissue with the sharpened end of each hook.

In some embodiments, each hook of the at least one hook has a blunt end, and attaching the surgical retractor comprises contacting the tissue with the blunt end of each hook.

In some embodiments, securing the tissue comprises manually holding the surgical retractor in place.

In some embodiments, securing the tissue comprises positioning the surgical retractor such that the surgical retractor is free standing while the tissue is retracted.

In some embodiments, the tissue is skin.

In some embodiments, the head comprises two hooks.

In some embodiments, the two hooks are spaced equidistant from the parallel axis.

In some embodiments, the head is offset to the left of the shaft.

In some embodiments, the head is offset to the right of the shaft.

In some embodiments, the head is offset anterior to the shaft.

In some embodiments, the distal portion is a square.

In some embodiments, the distal portion is curved.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is a view of a hook retractor, in accordance with one or more embodiments of this disclosure.

FIG. 2 is a side view of a hook retractor, in accordance with one or more embodiments of this disclosure.

FIG. 3 is a top view of a hook retractor, in accordance with one or more embodiments of this disclosure.

FIG. 4 is a side view of a hook retractor, in accordance with one or more embodiments of this disclosure.

FIG. 5 is a flowchart of an exemplary method of using a hook retractor, in accordance with one or more embodiments of this disclosure.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Surgical retractors help surgeons and operating room personnel hold an incision or wound open during a surgical procedure. Retractors hold back underlying organs and/or tissues, allowing surgeons greater visibility and access to the exposed areas. To address differing surgical needs for different surgical procedures, retractors are available in a variety of shapes and sizes.

Hook retractors are used to retract soft tissues, such as skin, most commonly in scar revision, wound closure, breast reconstruction, body contouring, excision of tumors, and facial lesions and cosmetic surgeries. A hook retractor can have either a single prong or a double prong for retracting the skin. In some embodiments, a hook retractor may help to prevent crushing tissue that would otherwise be harmed with the use of other tools. Generally, a hook retractor is held by a surgical assistant during the surgery, where the surgical assistant pulls the tissue back away from the surgeon to better expose the surgical area. The surgical assistant uses the hook to pull the skin away from the surgical site, which can be exhausting for the surgical assistant over the course of the surgical procedure and is a poor use of upper extremity biomechanics.

Embodiments of the present disclosure provide an ergonomically designed hook retractor that allows for pushing, rather than pulling, the soft tissue away from the surgical site. This design includes a retractor head facing the opposite direction as current retractor hooks, where the hooks face away from, rather than towards, the user. By reversing the angle of the hooks on the retractor head, this design allows the user to push skin or soft tissue away from the surgical site instead of pulling. Within this orientation, the hooks maintain the ability to pierce and solidly grip on an otherwise slick tissue. The inversion of the hooks allows for a much more ergonomic method of retraction, as the upper extremity extension motion of the user is generally stronger than the upper extremity flexion.

This design also allows for the retractor to be held by the surgeon, as opposed to a surgical assistant standing away from the surgeon. With the inverted hook retractor, the surgeon can hook the ends of the skin around the surgical site into a skin flap to elevate and push away from the surgical site. This design helps to maintain a clear surgical field while still retracting tissues away from the field of view.

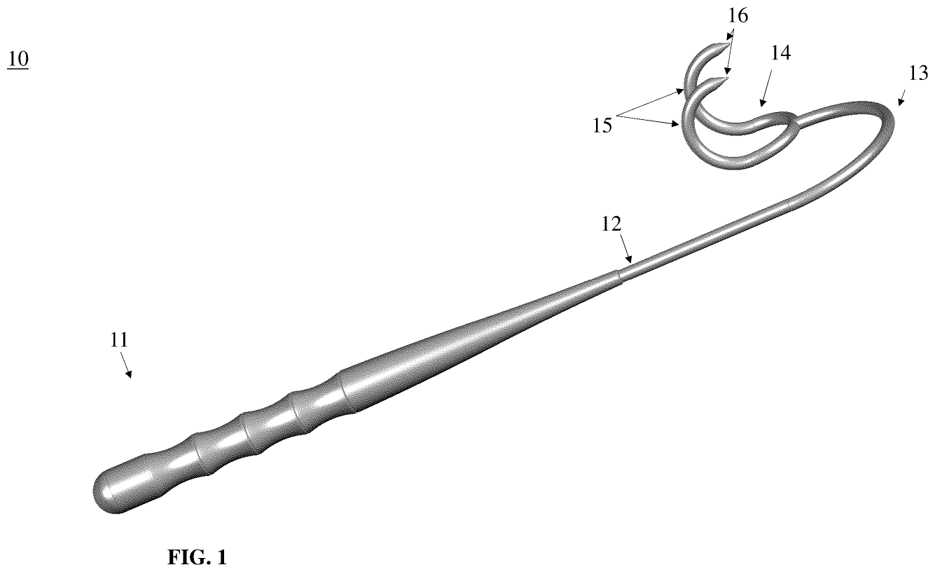

FIG. 1 is a view of an exemplary hook retractor, in accordance with one or more embodiments of this disclosure. The hook retractor 10 comprises a handle 11, a shaft 12, a curved portion 13, a head 14, and one or more hooks 15. A handle 11 is located at a proximal end of the hook retractor 10. Handle 11 may comprise one or more indentations to better fit the hand of the user. The proximal end of the handle 11 may be rounded. The distal end of handle 11 tapers towards a shaft 12 with a smaller diameter. The shaft 12 runs along a same axis as the handle 11 and extends distally along the axis towards a curved portion 13. The curved portion 13 of the shaft 12 is curved along a 180° arc, where an endpoint of the curved portion 13 lies along an axis parallel to that of the shaft 12 and handle 11. The final portion of the distal portion 13 from which the hook 15 originates is oriented antiparallel to the main shaft 12. In some embodiments, the distal portion 13 is squared off. In some embodiments, the diameter of the 180° arc of the curved portion is smaller such that the head 14 is closer to the shaft 12. In some embodiments, handle 11 is weighted to hold hook retractor 10 in place during a procedure. In certain embodiments, handle 11 can comprise further means for stabilization or placement, where the surgeon or user can push the tissue away from a surgical site to a desired location where it will remain in place during the procedure.

The head 14 of the hook retractor 10 is attached to the endpoint of the curved portion 13. The head 14 comprises, in the illustrated embodiment of FIG. 1, two hooks 15. It is contemplated that the head can comprise one hook or three or more hooks, as needed, with the understanding that the fewer or additional hooks are oriented in the same direction as hooks 15. Hooks 15 can be spaced apart from one another in a range of measurements, for example from 2 mm apart to 10 mm apart. It is contemplated that the hooks may be further apart or closer together in some embodiments. In some embodiments, the distance between the head 14 and the shaft 12 ranges from 2 to 6 cm. In some embodiments, the length of the shaft is between 8 and 16 cm. Regardless of component dimension, the geometric relationship between components is maintained throughout different embodiments.

The hooks 15 form 180° arcs, each culminating in an endpoint 16. In some embodiments, the endpoints 16 of hooks 15 are pointed, as shown in FIG. 1. In alternative embodiments, the endpoints 16 are blunt so as not to damage the soft tissues retracted from the surgical site. It is contemplated that the endpoints 16 can have a range of sharpened points, for use with a wide variety of procedures.

The head 14 is offset from the axis of the handle 11 and shaft 12 by the curved portion 13, such that the head 14 lies along a parallel axis. While the illustrated embodiment shows the head 14 offset from handle 11 and shaft 12 towards the left side of handle 11 and shaft 12, the head 14 is offset to the right side in other embodiments. These embodiments are designed to maximize comfort for either right-handed or left-handed users, without sacrificing the utility or the ergonomic design of the retractor. In practice, a right-handed user is likely to use the hook retractor 10 with their left hand, and a left-handed user with their right hand. In some embodiments, the head 14 is offset to be anterior to the handle 11 and shaft 12. Regardless of the orientation of the head 14 in relation to the handle 11 and shaft 12, the head 14 remains on an axis parallel to the axis of the handle 11 and shaft 12.

The hook retractor is made of carbon steel, stainless steel, aluminum, titanium, or any other suitable surgical tool material. While it is contemplated that the tool can be assembled from a number of individually manufactured pieces, the hook retractor is produced from a single cast piece of material for the sake of simplicity.

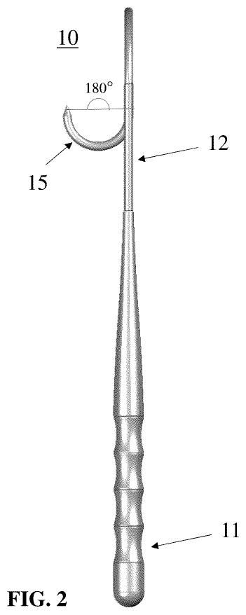

FIG. 2 illustrates a side view of the hook retractor 10. As discussed above, the head 14 and the handle 11 and shaft 12 lie on parallel axes. Accordingly, only the hook 15 is visible when the hook retractor 10 is viewed from the side as illustrated.

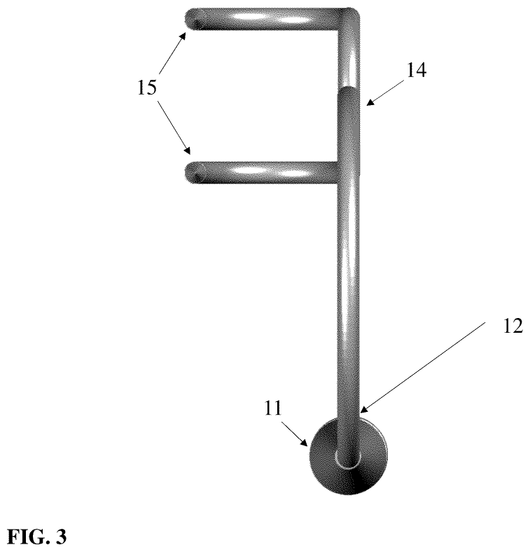

FIG. 3 illustrates a top view of the hook retractor 10. The top view of the hook retractor 10 illustrates the larger diameter of the handle 11 when compared to the diameter of the shaft 12. Further, the ends of each hook 15 are shown as facing the top view as well.

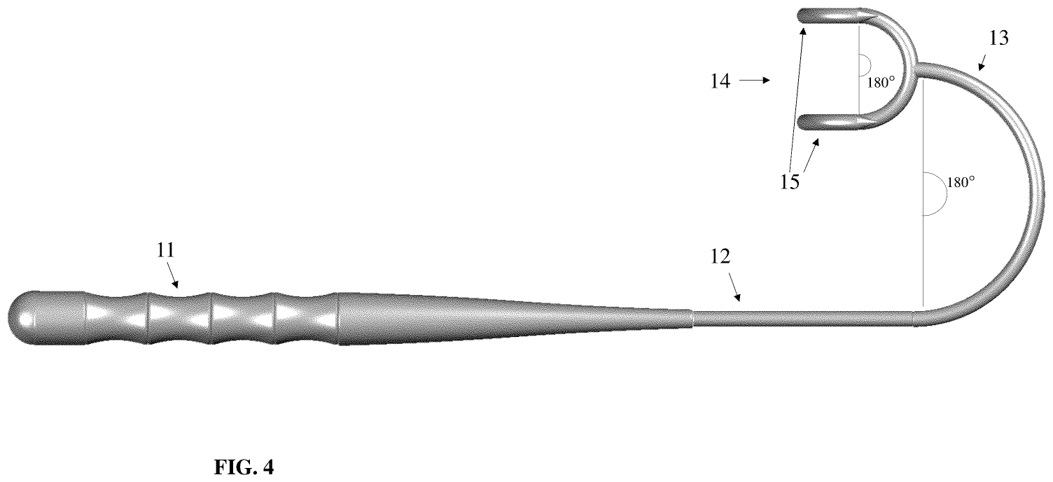

FIG. 4 illustrates an additional side view of the hook retractor 10. This view further illustrates how the handle 11 and shaft 12 are oriented along an axis parallel to axes containing the hooks 15.

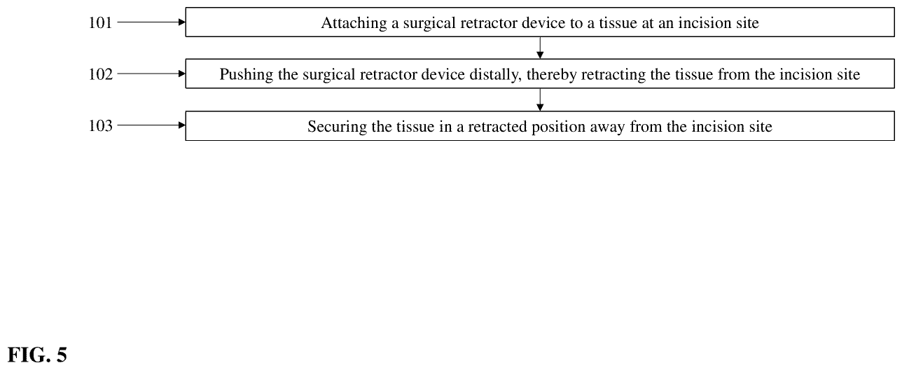

FIG. 5 is a flowchart of an exemplary method 100 for using a hook retractor. The exemplary method 100 (i.e., steps 101-103) may be performed by a surgeon or a surgical assistant during a surgery. In step 101, the method includes attaching a surgical retractor device to a tissue at an incision site. Attaching the retractor, such as, for example, hook retractor 10, can comprise piercing the tissue with a sharpened end of a hook, or securing a blunt end of a hook to a tissue. The surgical retractor device can comprise any of the embodiments discussed above, wherein the surgical retractor device comprises a handle, a shaft, and an inverted head offset from the handle and the shaft, the inverted head having a plurality of hooks.

In step 102, the method includes pushing the surgical retractor device distally, thereby retracting the tissue from the incision site. The user is able to effectively retract the tissue from the surgical site using less exertion than when the same tissue movement is achieved by pulling existing devices. The tissue can be retracted to any desired position by pushing, where the pushing is accomplished by either a surgeon or a surgical assistant during a surgical procedure.

In step 103, the method includes securing the tissue in or at a desired retracted position away from the incision site. Securing the tissue can be accomplished by continuing to push the retractor, or by holding the retractor in place while a surgical procedure is continued. Additionally, securing the tissue can be done by placing the retractor in a position such that the retractor holds the tissue away from the incision or surgical site without any additional action from the surgeon or surgical assistant.

In some embodiments, securing the tissue can comprise attaching the retractor to one or more arms. In some embodiments, the one or more arms can be mounted to a surgical bed or other fixed element.

In some embodiments, securing the tissue can comprise using one or more external anchors attached to the retractor.

In some embodiments, securing the tissue can comprise attaching the retractor to a frame. In some embodiments, the frame can comprise one or more arms coupled to a main bar.

In some embodiments, securing the tissue can comprise using a ratcheting mechanism.

In some embodiments, securing the tissue can comprise using one or more wheel fasteners.

In some embodiments, securing the tissue can comprise anchoring in bone or tissue.

In some embodiments, securing the tissue can comprise using a guidewire, a pedicle screw tower, a pin-type anchor, or other anchor.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A surgical retractor, comprising:
a handle with a central axis;
a shaft along the central axis with a distal portion; and
a head distal to the shaft, the head having at least one hook oriented such that an end of the at least one hook faces a direction opposite that of the handle,
wherein the head runs along a parallel axis to the central axis and is offset from the central axis.

2. The surgical retractor of claim 1, wherein the head comprises two hooks.

3. The surgical retractor of claim 2, wherein the two hooks are spaced equidistant from the parallel axis.

4. The surgical retractor of claim 1, wherein the end of the at least one hook is pointed.

5. The surgical retractor of claim 1, wherein the end of the at least one hook is blunt.

6. The surgical retractor of claim 1, wherein the head is offset to the left of the shaft or to the right of the shaft.

7. The surgical retractor of claim 1, wherein the head is offset anterior to the shaft.

8. The surgical retractor of claim 1, wherein the distal portion is angular.

9. The surgical retractor of claim 1, wherein the distal portion is curved.

10. A method of using a surgical retractor, comprising:
attaching a surgical retractor to a tissue at an incision site, the surgical retractor comprising:
a handle with a central axis;
a shaft along the central axis with a distal portion; and
a head distal to the shaft, the head having at least one hook oriented such that an end of the at least one hook is oriented to face a direction opposite that of the handle,
wherein the head runs along a parallel axis to the central axis and is offset from the central axis;
pushing the surgical retractor distally, thereby retracting the tissue away from the incision site; and
securing the tissue in a retracted position away from the incision site.

11. The method of claim 10, wherein each hook of the at least one hook has a sharpened end, and attaching the surgical retractor comprises piercing the tissue with the sharpened end of each hook.

12. The method of claim 10, wherein each hook of the at least one hook has a blunt end, and attaching the surgical retractor comprises contacting the tissue with the blunt end of each hook.

13. The method of claim 10, wherein securing the tissue comprises manually holding the surgical retractor in place.

14. The method of claim 10, wherein securing the tissue comprises positioning the surgical retractor such that the surgical retractor is free standing while the tissue is retracted.

15. The method of claim 10, wherein the tissue is skin.

16. The method of claim 10, wherein the head comprises two hooks.

17. The method of claim 16, wherein the two hooks are spaced equidistant from the parallel axis.

18. The method of claim 10, wherein the head is offset to the left of the shaft or to the right of the shaft.

19. The method of claim 10, wherein the distal portion is curved.

20. The method of claim 10, wherein the distal portion is angular.
